(19) United States
(12) Patent Application Publication
(10) Pub. No.: US 2025/0302892 A1
(43) Pub. Date: Oct. 2, 2025

(54) PROBIOTIC FOR USE IN THE TREATMENT OF INFLAMMATORY ALTERATIONS OF THE INTESTINAL MUCOSA, PARTICULARLY OBESITY

(71) Applicant: CERBIOS-PHARMA SA, Barbengo-Lugano (CH)

(72) Inventors: Raffaella COPPOLECCHIA, Barbengo-Lugano (CH); Luca ANTONIOLI, Pisa (PI) (IT); Matteo FORNAI, Pisa (PI) (IT); Carolina PELLEGRINI, Pisa (PI) (IT)

(21) Appl. No.: 18/865,311

(22) PCT Filed: Jun. 20, 2023
(86) PCT No.: PCT/EP2023/066533

§ 371 Date: Nov. 12, 2024

(30) Foreign Application Priority Data
Jun. 22, 2022 (IT) 102022000013138

(51) Int. Cl. A61K 35/744; A61K 31/19; A61K 35/00; A61P 3/04

(52) U.S. Cl. CPC A61K 35/744; A61K 31/19; A61P 3/04; A61K 2035/115

(57) ABSTRACT

The use of a probiotic, in particular an Enterococcus faecium strain named SF68, for the treatment of disorders characterized by alterations of the intestinal mucosa, particularly inflammations associated with altered intestinal permeability is described.

The present invention relates to the use of a probiotic for the treatment of disorders characterized by alterations of the intestinal mucosa, particularly inflammations associated with altered intestinal permeability, more specifically and preferably obesity.

In the last years, several clinical evidences have shown an alteration of the intestinal mucosa in several pathological conditions characterized by the presence of enteric inflammation, such as for example intestinal chronic inflammatory diseases (MICI), irritable bowel syndrome (IBS), obesity, but also in neurodegenerative pathologies such as Parkinson's disease and Alzheimer's disease [Genser L. et al., Increased jejunal permeability in human obesity is revealed by a lipid challenge and is linked to inflammation and type 2 diabetes. J. Pathol. 2018 October 246(2):217-230; Michielan A. et al., Intestinal Permeability in Inflammatory Bowel Disease: Pathogenesis, Clinical Evaluation, and Therapy of Leaky Gut. Mediators Inflamm. 2015, 2015:628157; Shulman et al., Associations among gut permeability, inflammatory markers, and symptoms in patients with irritable bowel syndrome, J. Gastroenterol. 2014 November, 49(11):1467-76; Sharma S. et al., Altered gut microbiota and intestinal permeability in Parkinson's disease: Pathological highlight to management. Neurosci. Lett. 2019 Nov. 1, 712:134516; Sochocka M. et al., The Gut Microbiome Alterations and Inflammation-Driven Pathogenesis of Alzheimer's Disease—a Critical Review, Mol Neurobiol. 2019 March, 56(3):1841-1851].

In particular, it has been observed that this increased intestinal permeability is connected to a reduced expression of the tight junction proteins, such as occludin, zonulin and claudin, which are essential elements to maintain the barrier integrity [Chelakkot C. et al., Mechanisms regulating intestinal barrier integrity and its pathological implications, Exp Mol Med. 2018 Aug. 16, 50(8):1-9]. Among the main systems involved in preserving the epithelial integrity, several pre-clinical and clinical studies have underlined the importance of butyric acid, a short chain fatty acid (SCFA) [Ma X. et al., Butyrate promotes the recovering of intestinal wound healing through its positive effect on the tight junctions, J Anim Sci. 2012 December, 90 Suppl 4:266-8, doi:10.2527/jas.50965, PMID:23365351; Yang T. et al., Amelioration of non-alcoholic fatty liver disease by sodium butyrate is linked to the modulation of intestinal tight junctions in db/db mice, Food Funct. 2020 Dec. 1, 11(12):10675-10689; Vargas-Robles H. et al., Beneficial effects of nutritional supplements on intestinal barrier functions in experimental colitis models in vivo, World J Gastroenterol. 2019 Aug. 14, 25(30):4181-4198; Gao Y. et al., Short chain fatty acid butyrate, a breast milk metabolite, enhances immature intestinal barrier function genes in response to inflammation in vitro and in vivo, Am J Physiol Gastrointest Liver Physiol. 2020 Oct. 21; Tabat et al., Acute Effects of Butyrate on Induced Hyperpermeability and Tight Junction Protein Expression in Human Colonic Tissues, Biomolecules. 2020 May 14, 10(5):766; Liu J. et al., Beneficial effects of butyrate in intestinal injury, J Pediatr Surg. 2020 June, 55(6):1088-1093].

This SCFA, mainly produced by the intestinal bacterial flora, performs a beneficial trophic effect on the intestinal epithelium, by stimulating the expression of the tight junctions [Ma X. et al., supra; Yang T. et al., supra; Vargas-Robles H. et al., supra; Gao Y. et al., supra; Tabat et al., supra; Liu J. et al., supra] and meanwhile performing an antiinflammatory effect by directly acting on several populations of immune cells [Bonomo R. R. et al., Fecal transplantation and butyrate improve neuropathic pain, modify immune cell profile, and gene expression in the PNS of obese mice, Proc Natl Acad Sci USA, 2020 Oct. 20, 117(42):26482-26493; Yang W. et al., Intestinal microbiota-derived short-chain fatty acids regulation of immune cell IL-22 production and gut immunity, Nat Commun., 2020 Sep. 8, 11(1):4457; Takahashi D. et al., Microbiota-derived butyrate limits the autoimmune response by promoting the differentiation of follicular regulatory T cells, EBioMedicine, 2020 August, 58:102913; Furusawa Y. et al., Commensal microbe-derived butyrate induces the differentiation of colonic regulatory T cells, Nature, 2013 Dec. 19, 504(7480):446-50, doi:10.1038/nature12721; Chen J. et al., The Role of Butyrate in Attenuating Pathobiont-Induced Hyperinflammation, Immune Netw. 2020 Feb. 4, 20(2):e15].

Several clinical evidences have shown that the presence of intestinal inflammation negatively affects the use of butyrate. In fact, it has been observed that patients with MICI, IBS or colon-rectal neoplastic disease are characterized by a reduced expression of butyrate transporters, which results in a deficient uptake of this SCFA with consequent deficiency at the enterocyte level and then increased intestinal permeability [Bonomo R. R. et al., supra; Yang W. et al., supra; Takahashi D. et al., supra; Furusawa Y. et al., supra; Chen J. et al., supra].

EP 2 289 505 discloses a nutritional composition containing probiotics, prebiotics and butyric acid or a salt thereof to alleviate intestinal disorders, particularly diarrhea and constipation.

Therefore, an improved use of butyrate would have a beneficial effect on patients suffering from disorders characterized by enteric inflammation and/or altered intestinal permeability.

We have now found that the treatment with a probiotic, in particular an Enterococcus faecium strain, more particularly an Enterococcus faecium strain with deposit number NCIMB 10415 which is called SF68© as probiotic active ingredient, increases the ability of the intestinal epithelial cells to use butyrate, by increasing its ability to express transporters, together with the improvement of the citrate synthase activity (key enzyme in the Krebs cycle, essential for a correct use of butyrate by the cells). By improving the use of butyrate, the probiotic SF68 is able to strengthen the intestinal barrier, through an increased expression of the tight junctions.

Therefore, object of the present invention is a supplement based on Enterococcus faecium, optionally in combination with butyric acid or salts thereof, for use in the treatment of diseases and disorders characterized by enteric inflammation and/or altered intestinal permeability, particularly obesity.

A supplement based on SF68 is preferably used. A pharmaceutical product based on SF68 is available on the market, particularly under the name Bioflorin® (Cerbios Pharma SA), and its efficacy and safety as probiotic is widely known (Holzapfel W. et al., Enterococcus faecium SF68 as a model for efficacy and safety evaluation of pharmaceutical probiotics, Beneficial Microbes, 2018, 9(3):375-388).

Butyric acid and its salts are available on the market in the form of food supplements as well. It is more commonly used in the form of sodium or calcium salt, that is as sodium butyrate or calcium butyrate, which are the preferred form for the combined use according to the present invention.

When the probiotic is administered in combination with butyric acid, the two components of the combination can be administered separately or in a single dosage form, preferably separately.

When they are administered separately, the two components of the combination can be administered simultaneously or sequentially or at a time difference one from the other.

Indicatively, but without any limitation, SF68 dosages are generally within the range 10⁷-10¹⁰, preferably not lower than 10⁹. For butyric acid and its salts, the dosage is generally in the range 200-500 mg, preferably not lower than 250 mg. The indicated dosages are generally administered 1-3 times a day for a period of at least one week. It is worth noting that the above reported dosages can vary depending on the disorder to be treated, its severity, on the patient's conditions, etc. The skilled in the art can easily identify the most suitable dosages outside the reported ranges, if he/she deems it necessary.

The use of probiotic SF68, optionally in combination with butyric acid or salts thereof, according to the present invention, has a beneficial effect on several diseases and disorders characterized by enteric inflammation and/or altered intestinal permeability. Among such diseases and disorders, obesity, enteropathy caused by NSAIDs, inflammatory bowel disease (IBD), irritable bowel syndrome (IBS) and diseases characterized by neuroinflammation at CNS level with resulting cognitive impairment are of particular interest, without limiting the scope of the present invention to those diseases/disorders.

The particular interest for these diseases and disorders within the scope of the present invention is due to the availability of animal models for the efficacy study as disclosed in more details herein after.

### Obesity

Overweight and obesity are a global public health issue. The incidence of obesity in western countries has remarkably increased and this results in the need of further studies to better understand the disease and its complications or co-morbidities. The animal models of diet-induced obesity (DIO) can reproduce human overweight and obesity. In fact, there are many protocols used to lead to excess fat accumulation in mice [de Moura E. et al., Diet-induced obesity in animal models: points to consider and influence on metabolic markers, Diabetol Metab Syndr., 2021 Mar. 18, 13(1):32]. In this respect, it is worth recalling that HFD (High-Fat Diet)-induced obesity is presently considered an important tool to understand the impact of the western diets with high fat content on the development of obesity and related disorders [Wang C. Y., et al., A mouse model of diet-induced obesity and insulin resistance, Methods Mol Biol. 2012, 821:421-433]. In fact, the intake of fat-rich diets in mice may lead to the development of human-type obesity, since body adiposity and leptin increase and cause the development of hypertension and glucose intolerance [de Moura E. et al., supra].

Based on the experience and on previous reports [Kim K. A. et al. High fat diet-induced gut microbiota exacerbates inflammation and obesity in mice via the TLR4 signaling pathway, PLoS One. 2012; 7(10):e47713; Laurila A. et al. High-fat, high-cholesterol diet increases the incidence of gastritis in LDL receptor-negative mice, Arterioscler Thromb Vasc Biol. 2001; 21(6):991-996], mice fed with HFD for 8 weeks showed a marked increase in body weight, followed by a marked alteration of several metabolic indexes, such as an increase in glycemia, cholesterol and triglycerides, so confirming the suitability of this experimental model. Recent studies also proved that HFD animals showed an increased expression of pro-inflammatory cytokines (TNF, IL-1β, IL-6) in the intestinal tissues, followed by spontaneous dysfunction of caliciform cells, altered mucin biosynthesis and damaged mucosal barrier [Kim K. A. et al., supra; Ding S. et al., Role of intestinal inflammation as an early event in obesity and insulin resistance, Curr Opin Clin Nutr Metab Care. 2011; 14:328-33; Gulhane M. et al., High fat diets induce colonic epithelial cell stress and inflammation that is reversed by IL-22, Sci Rep. 2016; 6:28990].

It is known that obesity is also associated with an early cognitive impairment, triggered by inflammatory processes of the CNS supported by an alteration of the blood-brain barrier [Miller A A, Spencer S J. Obesity and neuroinflammation: a pathway to cognitive impairment. Brain Behav Immun. 2014 November; 42:10-21; Rhea E M, Salameh T S, Logsdon A F, Hanson A J, Erickson M A, Banks W A. Blood-Brain Barriers in Obesity. AAPS J. 2017 July; 19(4):921-930].

### Enteropathy Caused by NSAIDs

The coming of new imaging techniques, such as video capsule endoscopy, allowed to obtain new information about the NSAID-induced intestinal damage, which appears to be site-specific. The mucosal injury type, which can be found in up to 75% NSAID consumers, ranges from damaged portions, mainly observed in the proximal small intestine, to mucosal erosions and ulcers in the distal portion.

The pathogenesis of the small intestine damage is not yet fully understood. The synthesis of mucosal endogenous prostaglandins is inhibited by NSAIDs through the entire gastrointestinal tract. However, other important pathogenic factors contributing to the damage, such as the presence of bacteria and bile which are essential triggering factors for the mucosal damage, can be different between the distal and proximal intestinal regions.

NSAIDs increase the intestinal permeability in patients, leading to a low degree intestinal inflammation, triggered by an increased permeability of the intestinal barrier followed by a marked bacterial infiltration. The increased intestinal permeability caused by the topical effect of NSAIDs is increased due to the inflammatory response (to luminal aggressors) and to the microvascular effects of the COX inhibition [Bjarnason I. et al., Mechanisms of Damage to the Gastrointestinal Tract From Nonsteroidal Anti-Inflammatory Drugs, Gastroenterology. 2018 February; 154(3):500-514]. The present model is mainly based on the results from mouse models, wherein some aspects of the damage show remarkable similarities with humans, such as the increase of the intestinal permeability observed with NSAIDs, the positioning of the enteropathy by NSAIDs at the medium-distal small intestine. As seen in the clinical studies, also in the preclinical model the onset of NSAID-induced dysbiosis and the damage of the intestinal mucosal barrier are the primum movens of a series of physiopathological events, which lead to the mucosal damage. In fact, the damaged tight junctions make easier the entry and the action of bacteria and bacterial antigens (as well as other luminal factors), which induce a higher expression and a higher release of pro-inflammatory cytokines from the intestinal epithelium [Colucci R. et al., Pathophysiology of NSAID-Associated Intestinal Lesions in the Rat: Luminal Bacteria and Mucosal Inflammation as Targets for Prevention, Front Pharmacol. 2018 Nov. 29; 9:1340; Fornai M. et al., Small bowel protection against NSAID-injury in rats: Effect of rifaximin, a poorly absorbed, GI targeted, antibiotic. Pharmacol Res. 2016 February; 104:186-96; Bjarnason I. et al., Mechanisms of Damage to the Gastrointestinal Tract From Nonsteroidal Anti-Inflammatory Drugs. Gastroenterology. 2018 February; 154(3):500-514].

### IBD/IBS Models

Intestinal bowel disease (IBD) is a group of inflammatory conditions of colon and small intestine caused by a dysregulated immune response. Chron disease (CD) and ulcerative colitis (UC) are the main types of IBD. Usually both involve severe diarrhea, pain, fatigue and weight loss. IBD can be debilitating and sometimes leads to potentially life-threatening complications.

An impaired intestinal barrier may cause an increase in intestinal permeability promoting the exposure to the luminal content and triggering an immune response which in turn promotes intestinal inflammation. The IBD patients show several defects of the specialized components of the mucosal barrier, from the composition of the mucus layer to the adhesins which regulate the cell permeability. These alterations may represent a primary dysfunction in Chron disease, but may also perpetuate the mucosal chronic inflammation in the ulcerative colitis [Michielan A. et al., Intestinal Permeability in Inflammatory Bowel Disease: Pathogenesis, Clinical Evaluation, and Therapy of Leaky Gut. Mediators Inflamm. 2015; 2015:628157].

A basic approach to the study of the pathogenesis and complexity of human IBD was the development of a variety of animal models. These animal models provided significant and essential in-depth analyses on histopathological and morphological changes in the intestinal tract linked to the human IBD pathogenesis. These models became an essential tool to explain the histopathological, immunological and morphological changes in the intestinal tract and potential therapeutic targets [Eichele D. D. et al., Dextran sodium sulfate colitis murine model: An indispensable tool for advancing our understanding of inflammatory bowel diseases pathogenesis. World J Gastroenterol. 2017 Sep. 7; 23(33):6016-6029].

Several experiments on animals during the last 25 years used the model of DSS (dextran sulphate)-induced colitis as chemical induction of the inflammation model morphologically and symptomatically resembling to the epithelial damage observed in human ulcerative colitis. In mice, colitis by DSS leads to a decrease of the tight junction expression, followed by an increase of the permeability and the clinical events of colon inflammation. In particular, the tight junction protein pattern undergoes quick changes, such as the increased claudin-2 expression and the decrease of several claudins and occludin-1. Therefore, the impairment of the mucosal barrier is seen as a secondary event to the increase of colon mucosal permeability with consequent afflux of inflammatory cells in the intestinal mucosa [Eichele D. D. et al., supra].

Irritable bowel syndrome (IBS) is a type of functional gastrointestinal disorder (FGID) characterized by symptoms such as abdominal pain or discomfort and irregularities in feces, not associated with metabolic or organic anomalies. Several factors are involved in the physiopathology of IBS, such as visceral sensitiveness, gastrointestinal (GI) motility, brain-intestine interaction and psychosocial stress. It is interesting to note that IBS frequently occurs in patients recovering from an infective colitis and IBS-like symptoms are often observed in patients with intestinal inflammatory disease (IBD) even after the intestinal inflammation has been removed.

IBS is underclassified as constipation-prevalent (IBS-C), diarrhea-prevalent (IBS-D) or with mixed symptoms (IBS-M). There is no therapy for IBS and the present treatment strategies often prescribe that the patients take several drugs to control their symptoms. In particular, the main clinical characteristics of IBS are alterations of the intestinal motility and the excretion and visceral pain [Kodani M. et al., Association between gastrointestinal motility and macrophage/mast cell distribution in mice during the healing stage after DSS-induced colitis. Mol Med Rep. 2018 June; 17(6):8167-8172].

The IBS experimental models may be divided into three types: animal model induced by a central stimulus, animal model induced by a peripheral stimulus and complex animal model induced by a combined central and peripheral stimulus. Among these models, the intestinal inflammation induced by chemicals such as DSS may induce IBS symptoms. In fact, mice recovered from colitis induced by DSS show a slower intestinal transit, a barrier dysfunction and a reduced EC cell density [Sharman S. K. et al., Sildenafil normalizes bowel transit in preclinical models of constipation. PLoS One. 2017 Apr. 27; 12(4):e0176673].

### Neurological Disorders

The microbiota-gut-brain axis is emerging as a key communication system involved in the maintenance of CNS homeostasis as well as in supporting the physiopathological events underlying several neurological disorders, such as mild cognitive impairment (MCI), dementia, multiple sclerosis (SM), Alzheimer (AD) and Parkinson (PD) disease and autism [Pellegrini C, Antonioli L, Colucci R, Blandizzi C, Fornai M. Interplay among gut microbiota, intestinal mucosal barrier and enteric neuro-immune system: a common path to neurodegenerative diseases?Acta neuropathologica. September 2018; 136(3):345-361. doi:10.1007/s00401-018-1856-5; Pellegrini C, Antonioli L, Calderone V, Colucci R, Fornai M, Blandizzi C. Microbiota-gut-brain axis in health and disease: Is NLRP3 inflammasome at the crossroads of microbiota-gut-brain communications?Progress in neurobiology. August 2020; 191:101806. doi:10.1016/j.pneurobio.2020.101806]. The mechanisms underlying the microbiota-gut-brain axis mainly depend on the interactions of intestinal bacteria and their metabolites with the intestinal epithelial barrier, the immune system and the ascending nervous fibers. In fact, it has been observed that pathogenic bacterial strains and their products can migrate in the blood flow and spread upward to the brain, where they may impair the blood-brain barrier integrity and affect the central neuronal circuit. In addition, pathogenic bacterial products may directly activate circulating immune/inflammatory cells, which, in turn, may migrate towards the CNS and alter the blood-brain barrier [Fung T C, Olson C A, Hsiao E Y. Interactions between the microbiota, immune and nervous systems in health and disease. Nature neuroscience. February 2017; 20(2):145-155. doi:10.1038/nn.4476; Rothhammer V, Mascanfroni I D, Bunse L, et al. Type I interferons and microbial metabolites of tryptophan modulate astrocyte activity and central nervous system inflammation via the aryl hydrocarbon receptor. Nature medicine. June 2016; 22(6):586-97. doi:10.1038/nm.4106; Rooks M G, Garrett W S. Gut microbiota, metabolites and host immunity. Nature reviews Immunology. May 27 2016; 16(6):341-52. doi:10.1038/nri.2016.42].

Therefore, alterations of microbiota and intestinal epithelial barrier, enteric immune/inflammatory responses, such as inflammosome activation, may be early events in neurological disorders which promote neuroinflammation and central neuroinflammation through the gut-brain [Pellegrini C, Antonioli L, Colucci R, Blandizzi C, Fornai M., supra; Ortiz G G, Pacheco-Moises F P, Macias-Islas M A, et al.

Role of the blood-brain barrier in multiple sclerosis. Archives ofmedical research. November 2014; 45(8):687-97. doi:10.1016/j.arcmed.2014.11.013; Zenaro E, Piacentino G, Constantin G. The blood-brain barrier in Alzheimer's disease. Neurobiology of disease. November 2017; 107:41-56. doi:10.1016/j.nbd.2016.07.007; AI-Bachari S, Naish J H, Parker G J M, Emsley H C A, Parkes L M. Blood-Brain Barrier Leakage Is Increased in Parkinson's Disease. Frontiers in physiology. 2020; 11:593026. doi:10.3389/fphys.2020.593026]. In this context, the manipulation of the intestinal microbiota with pre- or probiotics has been proposed as an useful therapeutic approach for preventing CNS diseases [Joseph J, Depp C, Shih P B, Cadenhead K S, Schmid-Schonbein G. Modified Mediterranean Diet for Enrichment of Short Chain Fatty Acids: Potential Adjunctive Therapeutic to Target Immune and Metabolic Dysfunction in Schizophrenia? Frontiers in neuroscience. 2017; 11:155. doi:10.3389/fnins.2017.00155; Sharma S, Taliyan R, Singh S. Beneficial effects of sodium butyrate in 6-OHDA induced neurotoxicity and behavioral abnormalities: Modulation of histone deacetylase activity. Behavioural brain research. Sep. 15 2015; 291:306-314. doi:10.1016/j.bbr.2015.05.052; van de Wouw M, Boehme M, Lyte J M, et al. Short-chain fatty acids: microbial metabolites that alleviate stress-induced brain-gut axis alterations. The Journal of physiology. October 2018; 596(20):4923-4944. doi:10.1113/JP276431].

### BRIEF DESCRIPTION OF THE FIGURES

FIG. 1—(A) Changes of body weight (%), (B) of epididymal fat weight, (C) of spleen weight and (D) of liver weight in mice fed with SD+lactose, SD+SF68, HFD+lactose and HFD+SF68 in the same 4-week (4 w), 4+4 week (4+4 w) and 8 week (8 w) therapeutic scheme (**P<0.0001 vs SD, *P<0.05 vs SD, ^{aa}P<0.0001 vs HFD, ^{a}P<0.05 vs HFD).

FIG. 2—Levels of total cholesterol (A), HDL (B), LDL (C) and (D) HDL/LDL ratio in mice fed with SD+lactose, SD+SF68, HFD+lactose and HFD+SF68 in the 4 w, 4+4 w or 8 w therapeutic scheme (****P<0.0001 vs SD, ***P<0.0005 vs SD, *P<0.05 vs SD, ^{aa}P<0.0001 vs HFD)

FIG. 3—Levels of triglycerides (A) and glycate hemoglobin (Hb) (B) in mice fed with SD+lactose, SD+SF68, HFD+lactose and HFD+SF68 in the 4 w, 4+4 w or 8 w therapeutic scheme (****P<0.0001 vs SD, ***P<0.0005 vs SD, *P<0.01 vs SD, ^{aa}P<0.0001 vs HFD)

FIG. 4—Levels of IL-1p (A) and LBP (B) in mice fed with SD+lactose, SD+SF68, HFD+lactose and HFD+SF68 in the 4 w, 4+4 w or 8 w therapeutic scheme (**P<0.001 vs SD, *P<0.05 vs SD, ^{a}P<0.05 vs HFD).

FIG. 5—Fecal levels of butyrate (A) and propionate (B) in mice fed with SD+lactose, SD+SF68, HFD+lactose and HFD+SF68 in the 4 w, 4+4 w or 8 w therapeutic scheme (*P<0.05 vs SD).

FIG. 6—Fecal levels of calprotectin in mice fed with SD+lactose, SD+SF68, HFD+lactose and HFD+SF68 in the 4 w, 4+4 w or 8 w therapeutic scheme (***P<0.0001 vs SD).

FIG. 7—Tissue levels of MPO (A), MDA (B) and IL-1β (C) in mice fed with SD+lactose, SD+SF68, HFD+lactose and HFD+SF68 in the 4 w, 4+4 w or 8 w therapeutic scheme (**P<0.001 vs SD, *P<0.05 vs SD, ^{a}P<0.05 vs HFD, ^{aa}P<0.0001 vs HFD).

FIG. 8—Colonic contractible responses induced in vitro by electrical stimulation or by stimulation with carbachol or P substance in mice fed with SD+lactose, SD+SF68, HFD+lactose and HFD+SF68 for 4 w.

FIG. 9—Colonic contractible responses induced in vitro by electrical stimulation or by stimulation with carbachol or P substance in mice fed with SD+lactose, SD+SF68, HFD+lactose and HFD+SF68 for 8 w (**P<0.001 vs SD, *P<0.05 vs SD, ^{a}p<0.05 vs HFD).

FIG. 10—Densimetric analysis of the expression of (A) zonulin, (B) occludin and (C) claudin-1 in mice fed with SD+lactose, SD+SF68, HFD+lactose and HFD+SF68 in the 4 w, 4+4 w and 8 w therapeutic scheme (*P<0.05 vs SD, ^{a}P<0.05 vs SD).

FIG. 11—Densimetric analysis of the expression of (A) TLR2 and (B) TLR4 in mice fed with SD+lactose, SD+SF68, HFD+lactose and HFD+SF68 in the 4 w, 4+4 w and 8 w therapeutic scheme (*P<0.05 vs HFD).

FIG. 12—Densimetric analysis of the expression of (A) NF-kB and (B) MyD88 in mice fed with SD+lactose, SD+SF68, HFD+lactose and HFD+SF68 in the 4 w, 4+4 w and 8 w therapeutic scheme (*P<0.05 vs SD, ^{a}P<0.05 vs HFD).

FIG. 13—Densimetric analysis of the expression of claudin-1 in mice fed with SD+lactose, SD+SF68, HFD+lactose and HFD+SF68 in the 4 w, 4+4 w and 8 w therapeutic scheme (***P<0.0001 vs SD, **P<0.001 vs SD, ^{aaa}P<0.002 vs HFD, ^{a}P<0.05 vs HFD).

FIG. 14—Densimetric analysis of the inflammatory infiltrate in mice fed with SD+lactose, SD+SF68, HFD+lactose and HFD+SF68 in the 4 w, 4+4 w and 8 w therapeutic scheme (**P<0.001 vs SD, ^{aaa}P<0.002 vs HFD, ^{aa}P<0.001 vs HFD).

FIG. 15—Representative blots and densimetric analysis of the expression of (A) SMCT1, (B) MCT1 and (C) MCT4 in mice fed with SD+lactose, SD+SF68, HFD+lactose and HFD+SF68 in the 4+4 w and 8 w therapeutic scheme (**P<0.01 vs SD, ^{a}P<0.0001 vs HFD).

FIG. 16—Analysis of citrate synthase activity in intestinal tissues (A) and fat tissue (B) in mice fed with SD+lactose, SD+SF68, HFD+lactose and HFD+SF68 in the 4+4 w and 8 w therapeutic scheme (*P<0.05 vs SD, ^{§} P<0.05 vs HFD).

FIG. 17—Plasma levels of homocysteine in mice fed with SD+lactose, SD+SF68, HFD+lactose and HFD+SF68 in the 4 w or 8 w therapeutic scheme (*P<0.05 vs HFD).

FIG. 18—TNF tissue levels at the brain in mice fed with SD+lactose, SD+SF68, HFD+lactose and HFD+SF68 in the 8 w therapeutic scheme.

The present invention is based on the efficacy of SF68 to counteract several aspects of the intestinal inflammation/alteration and on its ability to re-uptake butyrate. Based on these experimental observations, the administration of SF68, optionally in combination with butyric acid or a salt thereof, is particularly effective for the treatment of disorders characterized by altered intestinal permeability, including obesity, but are not limited to it.

In particular, the combined use allows to combine the beneficial effects of both SF68 and butyrate on the intestinal mucosa.

The efficacy of SF68 has been proved by experiments on mice treated with a high-fat diet (see Experimental Section).

In fact, from the obtained results it appears that:
the supplementation with SF68 in the 4 w, 4+4 w and 8 w administration schemes showed the ability to significantly counteract weight gain in animals fed with high-fat diet (HFD);
as for the metabolic parameters, HFD resulted in a significant increase of total cholesterol, LDL and triglycerides and in a decrease of HDL levels. The treatment with SF68 for 8 weeks significantly counteracted such changes;
animals fed with HFD show an increase in circulating levels of IL-1β and LPS binding protein (LBP, index of increased intestinal permeability). The supplementation with SF68 at 8 w significantly reduced such changes;
from the analysis of feces obtained from animals fed with HFD it appears that calprotectin levels increased and did not undergo significant modifications after a treatment with SF68;
as for the SCFA levels, the HFD does not change the fecal propionate levels. However, in HFD animals we note a significant increase of fecal butyrate levels. The butyrate increase is not seen in HFD animals treated with SF68 at 8 weeks.
analyzing the levels of tissue inflammatory parameters (IL-1β and MPO) and tissue oxidative stress (MDA), we note an increase of said parameters in animals fed with HFD. The supplementation with SF68 at 8 weeks resulted in a significant decrease of the abovementioned parameters, showing an antiinflammatory and antioxidant activity of the treatment;
from experiments carried out in vitro we can note that animals fed with high-fat diet show a modification of the neuronal coding at the colonic enteric nervous system, with a decrease of the cholinergic component and an increase of the tachykinergic tone. Such a modification is widely known to be at the basis of motor impairments observed in several pathologies characterized by the presence of intestinal inflammation. It is of upmost interest the effect of SF68 in counteracting such modifications, by preserving the cholinergic component and significantly antagonizing the excessive tachykinergic transmission. Such beneficial effect may be attributed to the decrease of the levels of inflammation and oxidative stress at the colonic tissue or to a direct effect on neuronal coding or to both;
animals fed with HFD show a marked decrease in the tight junction expression at the colonic tissue, proving a laxity of the intestinal barrier. The treatment with SF68 showed the ability to strengthen the barrier by increasing the tight junction expression;
animals fed with high-fat diet for 8 weeks showed a significant increase of TLR-4 levels at the colonic tissues. Such increases have been antagonized by the supplementation with SF68 for 8 weeks. Concurrently, by exploring intracellular paths related to TLR-4, we observed an increase of NFK-b after 8 weeks of high-fat diet. In the 4+4 w and 8 w scheme, SF68 antagonized such an increase;
the experiments to evaluate the effects of the probiotic on the system for the transport and use of butyrate led to observe a reduced uptake ability of this SCFA in obese animals with reduced expression of butyrate transporters and a reduced citrate synthase activity. SF68 results in an improvement in the use of this SCFA by the intestinal mucosa, by restoring the levels of SMCT1 transporter and by normalizing the citrate synthase activity at the intestinal tissues;
SF68 positively affects the enteric homeostasis, resulting in a better use of butyrate, as experimentally proved through an increase of butyrate transporters and, by improving the citrate synthase activity, results in an improvement of the intestinal epithelium trophism, evidence also confirmed by a better tight junction expression, with consequent lower permeation of luminal antigens and then lower inflammatory load, as inferred by the pro-inflammatory cytokine decrease.
in addition, the treatment with SF68 resulted in improved tight junction expression at the CNS, with consequent reduction of the presence of the central inflammation condition (TNF level decrease).

In conclusion, the treatment with SF68 highlighted the ability to counteract the alterations of several systemic and tissue parameters consequent to feeding with high-fat diet. The beneficial effects of SF68 on the intestinal barrier trophism have been observed showing its strengthening, as proved by the tight junction increase to be attributed to the SF68 ability to make the intestinal mucosa more sensitive to the uptake and use of butyrate, SCFA known for its beneficial abilities on the enteric mucosa trophism and on the intestinal immune system. All this appears to occur because of an antiinflammatory effect mediated by a positive effect on the intestinal barrier integrity. It has been observed that the positive effect on the tight junctions is related to an improved butyrate use/re-uptake by the enterocytes, as proved by the histochemical study of the inflammatory infiltrate, by the decrease of circulating LPS, IL-1β and TLR4, in addition to the decrease of NF-kB and MPO (signal of a reduced infiltrate of inflammatory cells).

This effect is of particular relevance in the context of the present invention, proving the efficacy of the probiotic, optionally in combination with butyric acid (or salts thereof), for the treatment of diseases and disorders characterized by enteric inflammation and/or altered intestinal permeability, including obesity.

Without being bound to any theory, based on the results on mice, we can assume that SF68 leads to a normalization of the expression of the apical transporter of butyrate, altered by the high-fat diet (HFD).

The utility, efficacy and advantages of the present invention will be now illustrated in greater details by the following examples which however are not intended to limit in any way the scope of the present invention.

### EXPERIMENTAL SECTION

Five-week old C57BL/6 mice (20-22 g weight) were provided by Envigo srl (San Pietro al Natisone, Udine, Italy). The mice were housed six in a cage in a temperature-controlled room on a 12-hour light cycle at 22-24° C., and 50-60% humidity and let familiarize for at least one week. The animals were handled according to the Directive 2010/63/UE.

Standard diet (SD, 18% calories from fat; TD.2018) was administered during the adaptation period to all mice. Then, the animals were randomly divided into six groups, each composed by 10 mice, as follows:

### Protocol A

Group A1: SD+vehicle with lactose (L) for 4 weeks (4 w)
Group A2: HFD+vehicle with lactose for 4 w
Group A3: SD+vehicle for 4 w+SF68 (P) starting from the fourth week
Group A4: SD+vehicle for 4 w+vehicle with lactose starting from the fourth week
Group A5: HFD+vehicle for 4 w+SF68 starting from the fourth week
Group A6: HFD+vehicle for 4 w+vehicle with lactose starting from the fourth week

### Protocol B

Group B1: SD+SF68 for 4 w
Group B2: HFD+SF68 for 4 w
Group B3: SD+SF68 for 8 weeks (8 w)
Group B4: HFD+SF68 for 8 w

The vehicle was 150 μL of 3% methocel.

SF68 was used as BioFlorin®.

The high-fat diet (HFD) provided 60% calories from fat (TD.06414).

The comparison between the calorie counts of the two diets is reported in the following Table 1:

TABLE 1
comparison between the calorie counts of SD and HFD

| | Kcal from | Kcal from | Kcal from | |
|---|---|---|---|---|
| Diet | carbohydrates | proteins | fats | |
| SD | 58% | 24% | 18% | (3.1 kcal/g) |
| HFD | 21.4% | 18.3% | 60.8% | (5.1 kcal/g) |

The body weights of the animals were measured once a week from the first day of the study and the percent changes of the body weights, depicted by taking as 100 the value of the animals of SD+L group, are reported in the following Table 2.

TABLE 2
Body weight percent changes in animals under testing

| | SD + | | | HFD + | |
|---|---|---|---|---|---|
| | L/SD + | SD + | HFD + | L/HFD + | HFD + |
| WEEK | SF68 | SF68 | L | SF68 | SF68 |
| 1 | −1.32% | −5.66% | +5.72% | +6.28% | +10.37% |
| 2 | +1.01% | −15.24% | +10.42% | +12.75% | +5.11% |
| 3 | +2.00% | −20.04% | +15.44% | +16.70% | +10.85% |
| 4 | +2.00% | −18.18% | +23.03% | +27.40% | +11.80% |
| 5 | +2.60% | −15.43% | +27.41% | +22.07% | +22.30% |
| 6 | +5.90% | −12.40% | +36.89% | +27.67% | +33.54% |
| 7 | +5.41% | −18.22% | +48.45% | +35.35% | +38.67% |
| 8 | +1.9% | −15.57% | +55.16% | +46.21% | +47.15% |

The mice fed with high-fat diet (HFD) showed a significant body weight increase after 4 and 8 weeks compared to the animals fed with normocaloric diet (SD). The treatment with SF68 significantly counteracted the weight increase induced by HFD after 4 as well as 8 weeks. The treatment with SF68 did not result in changes of the weight gain in animals fed with SD in all the tested treatment schemes.

At the end, the animals were anaesthetized and sacrificed. Blood samples and tissue samples were collected and stored ad −80° C. for further analysis.

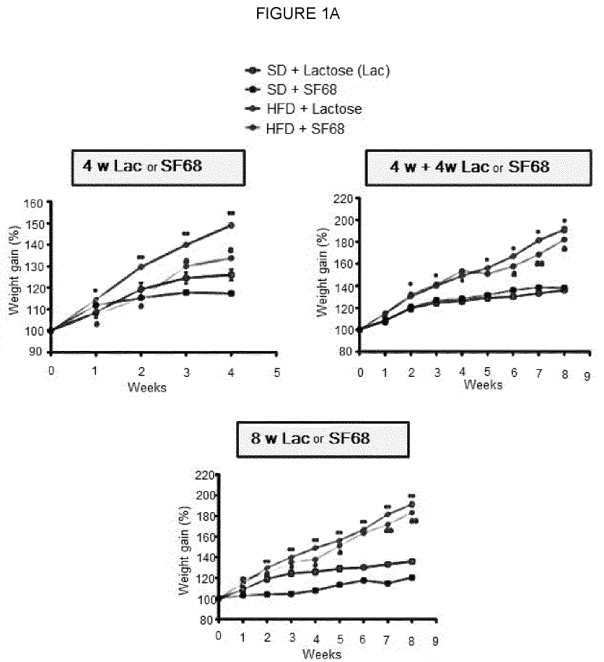

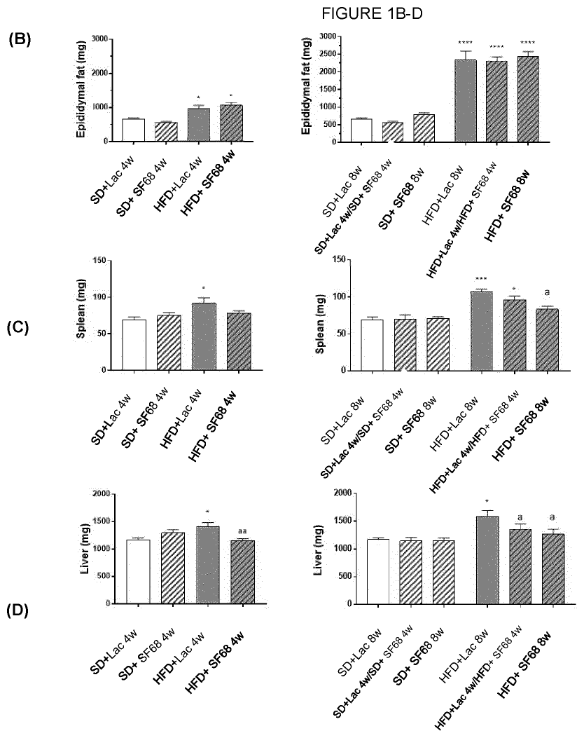

Changes of Body Weight and Organs The mice fed with high-fat diet (HFD) showed a significant body weight increase after 4 and 8 weeks compared to the animals fed with normocaloric diet (SD) (FIG. 1A). The treatment with SF68 significantly counteracted the weight increase induced by HFD after 4 as well as 8 weeks (FIG. 1A). The treatment with SF68 did not result in changes of the weight gain in animals fed with SD in all the tested treatment schemes (FIG. 1A).

In addition, in FIG. 1B-D the changes of epididymal fat weight (B), spleen weight (C) and liver weight (D) in mice fed with SD+lactose, SD+SF68, HFD+lactose and HFD+SF68 on the 4 week (4 w), 4+4 week or 8 week (8 w) therapeutic scheme are reported.

There is no significant effect on the fat accumulated in the epididymis. This fact is not anomalous since in HFD models the epididymal fat is the last to increase and likely the 8 weeks of the study did not allow to change this parameter.

On the contrary, a decrease in the weight of the spleen which, being a lymphoid organ, increases in weight in the presence of systemic inflammation, has been observed. A decrease in spleen weight is indicative of an antiinflammatory effect.

Normally, in HFDs the fatty tissue makes the liver fat (non alcoholic hepatic syndrome—NASH) and then less functional. A decrease in liver weight has been observed, and mainly a better appearance of HFD liver at the morphological examination in HFD animals treated with SF68 compared to those untreated suggests a protective effect by SF68 (smaller areas of fatty infiltration).

### Metabolic Parameters

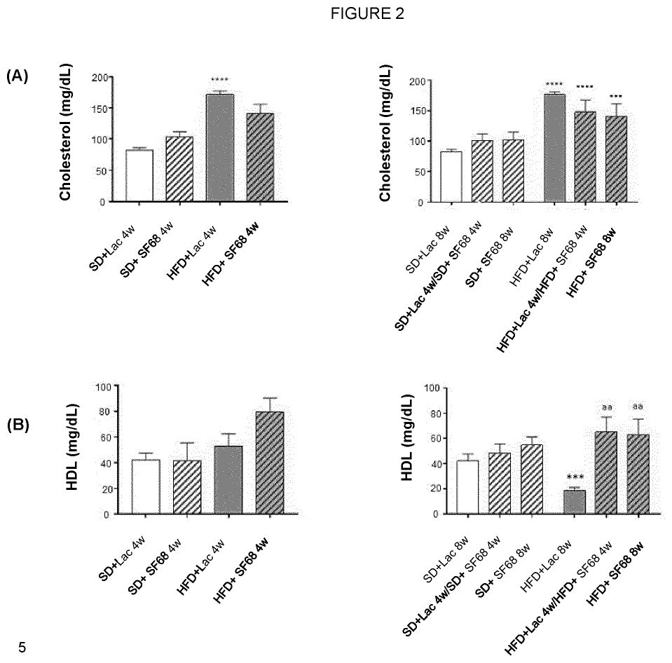

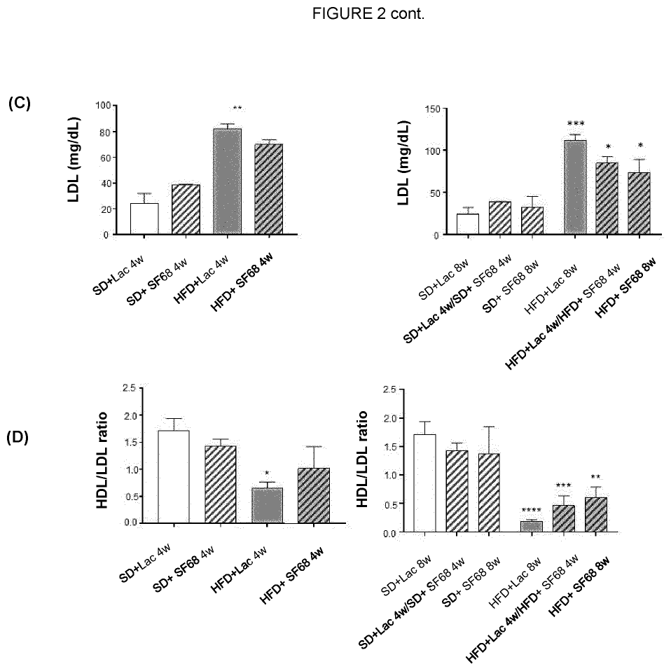

Animals fed with HFD showed a significant increase of plasma levels of total cholesterol (FIG. 2A), LDL (FIG. 2C) and a decrease of HDL (FIG. 2B) compared to mice fed with SD (FIG. 2A-D). The treatment with SF68 in mice under hypercaloric diet resulted in a significant increase of HDL levels compared to HFD mice (FIG. 2B), without changing the levels of total cholesterol (FIG. 2A) and LDL (FIG. 2C). Then, the HDL/LDL ratio has been favorably changed.

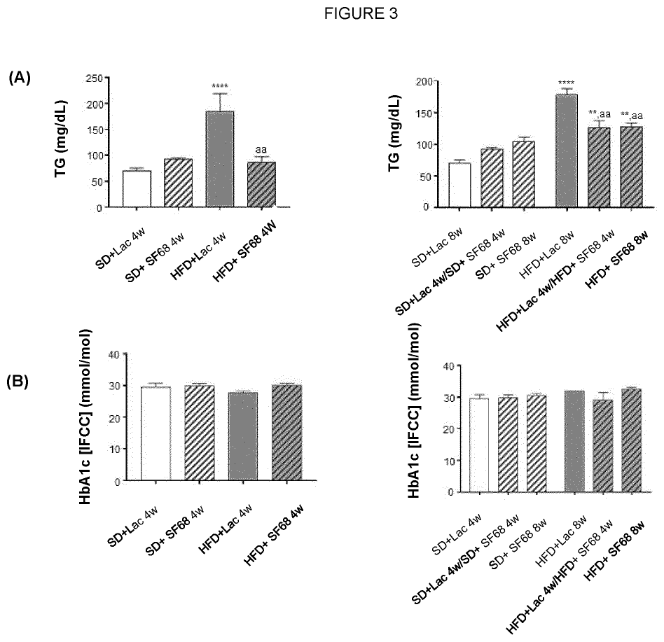

Animals fed with HFD showed a significant increase of plasma levels of triglycerides (FIG. 3A) compared to mice fed with SD (FIG. 3A) while the hemoglobin (Hb) levels do not appear to be altered (FIG. 3B). The treatment with SF68 in mice under hypercaloric diet resulted in a significant increase of triglyceride levels compared to HFD mice (FIG. 3A), without changing the levels of glycate Hb (FIG. 3B).

The lacking change of glycate Hb was predictable since in HFD models the increase is observed only at week 10-12, that is in a time period longer than the duration of this study.

Conversely, it is important the effect of decreasing triglycerides by SF68 which counteracts the effect of increasing triglycerides by HFD. The triglyceride increase is, together with the transaminase increase, associated with hepatic problems and therefore the observed decrease is favorable to a more healthy (also morphologically less infiltrated) liver.

### Plasmatic Levels of IL-1β and LBP (LPS Binding Protein)

The levels of IL-1β and LBP in plasma have been measured by ELISA (Prodotti Gianni, Milan, Italy), as described by Antonioli L. et al. [Int. J. Obes. (2019), doi:10.1038/s41366-018-0166-2]. For the procedure, blood samples were centrifugated for 5 minutes at 4000 rpm at 2-8° C.; after the centrifugation, the supernatant was collected. Aliquots (100 μl) were used for the analysis. The IL-1β levels were expressed in pg/ml of plasma, while the LBP levels were expressed in ng/ml of plasma.

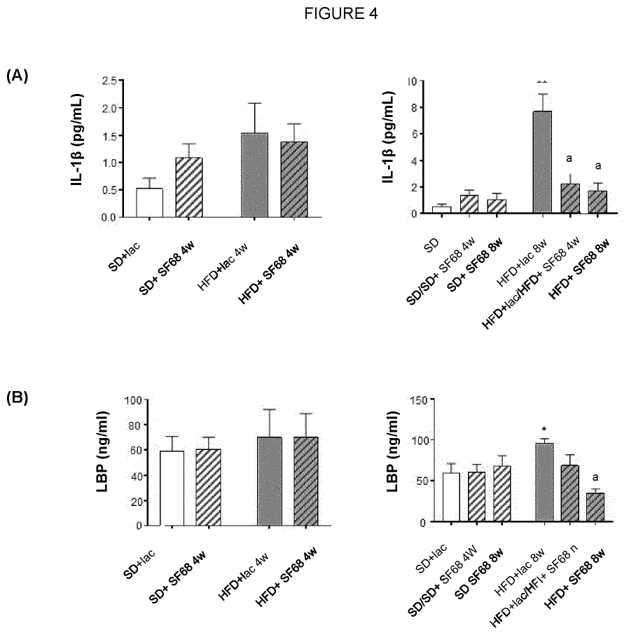

Animals fed with HFD showed a significant increase of plasma levels of IL-1β (FIG. 4A) and LBP (FIG. 4B) after 8 weeks of feeding with HFD compared to mice fed with SD (FIGS. 4A and B). The treatment with SF68 in mice under hypercaloric diet resulted in a significant increase of IL-1β levels in HFD mice in the 4+4 and 8 week scheme (FIG. 4A), while a decrease of circulating LBP levels was observed after only 8 weeks of treatment with SF68 (FIG. 4B).

IL-1β is pyrogenic, proinflammatory and triggers macrophage activation. The decrease of IL-1β plasma levels is in direct correlation with the antiinflammatory effect of the treatment with SF68.

When the intestinal epithelial barrier is damaged, intestinal microorganisms can more easily migrate into the blood and the same microorganisms (if gram-) and/or the liposaccharides (LPS) produced by their degradation are bound to LBP protein, which production by the hepatocytes remarkably increases when there are microorganisms in the blood. The significant LBP increase in mice fed with HFD indirectly suggests a reduced function of the intestinal epithelial barrier. Such increase is significantly counteracted by SF68 at 8 weeks of treatment and is indicative of the decrease in circulating LPS.

### Evaluation of Fecal Contents of SCFA

For the analysis of short chain fatty acids (SCFA), fecal samples were lyophilized, and gas chromatography was performed. The lyophilizate was solubilized in 100 ml 5 M formic acid and 400 ml acetone and centrifuged (5 min at 4000 rpm). Using a GC2010 Plus gas chromatograph (Shimadzu Deutschland GmbH, Duisburg, Germany) equipped with a flame ionization detection with a thin-film capillary column Stabilwax® (Restek, Bad Homburg, Germany), the concentration of SCFA in the supernatants was determined. The samples were spread out by split injection using the auto-sampler AOC-20s/l (Shimadzu Deutschland GmbH). GC Solution Chromatography Data System (Shimadzu Deutschland GmbH) was used for data processing. For the quantification of SCFA an external standard (Supelco™ WSFA-1 Mix, Supelco Sigma-Aldrich Co., Bellefonte PA) was employed.

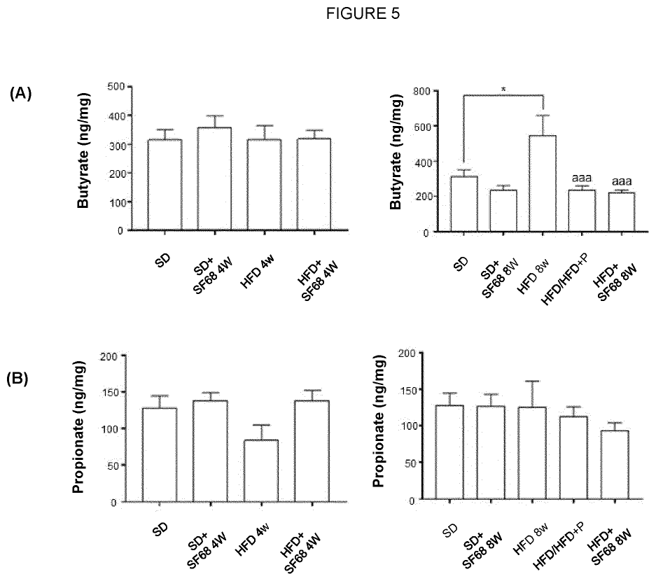

The mice fed with high-fat diet (HFD) showed a significant increase in fecal butyrate levels after 8 weeks of feeding with HFD (FIG. 5A). Such an increase resulted to be antagonized by the supplementation with SF68 in the 4+4 and 8 w therapeutic scheme. No changes were observed on the propionate levels (FIG. 5B).

The statistically significant information on SF68 was examined in depth in relation to the mechanism of action, with studies on intestinal butyrate transporters, and it was found that SF68 leads to normalize the expression of the butyrate apical transporter SMCT1, altered by HFD. A possible mechanism of action of SF68 may be the improvement of the intestinal epithelium health, for example through an improvement of the tight junctions and, mainly, with a higher expression of the SFCA transporters, which promote butyrate re-uptake and then its better use by the organism, both as trophic and antiinflammatory factor for the intestinal cells. Further studies, described hereinafter, allowed to observe that the treatment with SF68 is also associated with an increase of citrate synthase enzymatic activity (both intestinal and of fatty tissue). This enzyme catalyzes the first reaction of Krebs cycle, key process of the cell respiration, able to regulate the flow rate of the whole cycle and allowing the use of butyrate in the enterocytes. A hypercaloric diet results in a decrease of citrate synthase enzymatic activity, which is counteracted by SF68. This effect, in addition to that of improving the expression of SCFA transporters (and strengthening tight junctions), may explain how SF68 administration is associated with a more healthy intestine.

### Fecal Levels of Calprotectin

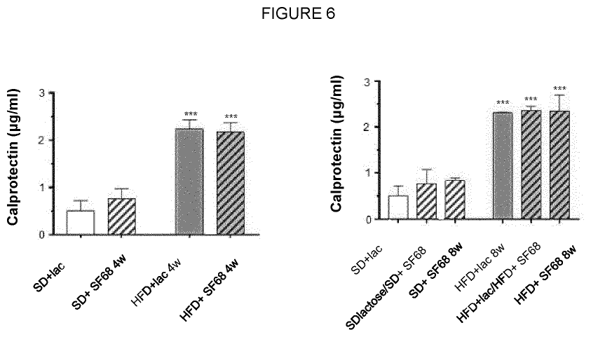

The mice fed with high-fat diet (HFD) showed a significant increase in the fecal calprotectin levels, a protein belonging to the S100 family present in high amount in neutrophil granulocytes. The treatment with SF68 did not significantly change such a parameter (FIG. 6).

This means that the antiinflammatory mechanism of SF68 cannot be explained through its direct effect on neutrophils.

### Myeloperoxidase (MPO), Malondialdehyde (MDA) and IL-1β Levels in Intestinal Tissues

The levels of MPO in colon tissues were measured by ELISA (Prodotti Gianni, Milan, Italy), as described by Antonioli L. et al. [FASEB J. (2020) 34, 5512-5524]. Colon samples previously stored at −80° C. were homogenized on ice with a Polytron Homogenizer (Qiagen, Milan, Italy). The homogenates were centrifuged at 4° C. for 15 min at 12000 rpm. Aliquots (100 μL) of supernatant were then used for the analyses. MPO levels were expressed in ng/mg tissue.

MDA concentration in intestinal samples was measured to obtain a quantitative estimation of the mucosal infiltration of polymorphonuclear cells. The analysis was carried out as previously described by Antonioli et al. [Int. J. Obes. (2019), supra]. Intestinal tissues were weighed, grinded and homogenized with a Polytron homogenizer in 2 ml of cold buffer (QIAGEN, Milan, Italy) and centrifuged at 1200 rpm for 10 min at 4° C. MDA concentrations were measured by using a kit for colorimetric assay (Calbiochem, San Diego, CA, USA), and the results expressed in nmoles MDA per mg colonic tissue.

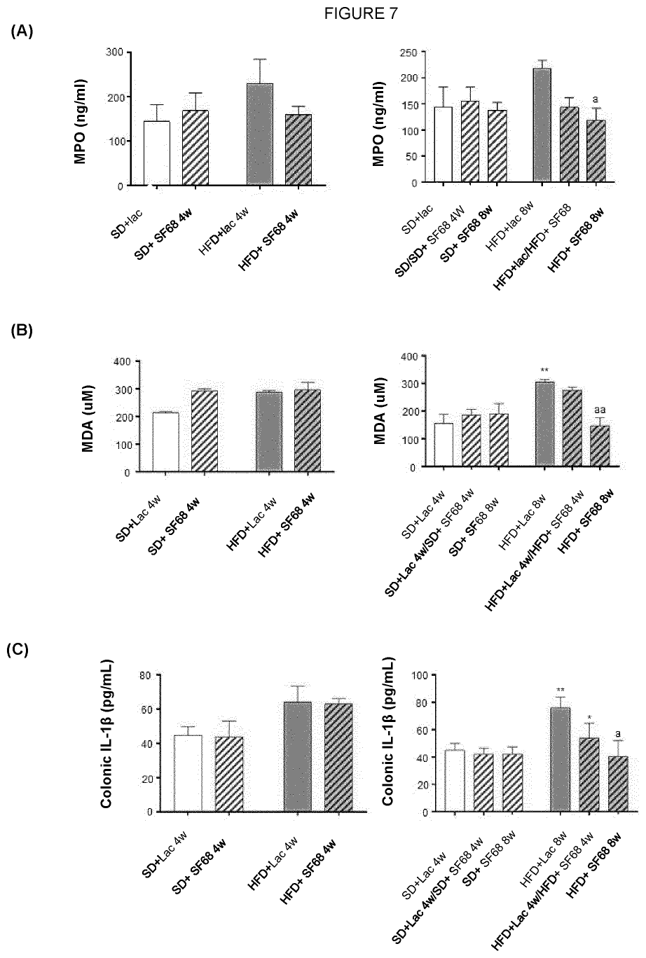

In colonic tissue samples isolated from mice fed with a fat diet, a significant increase of MPO, MDA and IL-1β levels was observed (FIGS. 7A, B and C) compared to what observed in tissues taken from animals fed with SD (FIGS. 7A, B and C). In HFD mice, the treatment with SF68 resulted in a significant decrease of MPO, MDA and IL-1β levels (FIG. 7) compared to what observed in HFD mice not receiving a diet supplementation with SF68.

In tissues from mice fed with HFD, significant increases of MPO, coming from infiltrates of immuno-inflammatory cells, MDA, which is a signal of oxidative stress, and IL-1β, pyrogenic and proinflammatory cytokine, were observed. The treatment with SF68 at 8 weeks resulted in a significant decrease of these three components in tissues. MDA reduction, which means less oxidative stress, could be due to an effect of SF68 as a support for endogenous scavangers (such as, e.g., glutathione). MPO reduction means less infiltrate of inflammatory cells. IL-1β reduction confirms the antiinflammatory effect of SF68, in fact a decrease of this interleukine was observed also in the blood.

### In Vitro Evaluation of Colonic Contractile Activity

The contractile activity of colonic muscle preparations was carried out as described by Antonioli et al. [FASEB J., supra], with minor changes. Following sacrifice, the colon was immediately removed by an incision above the anal end and placed into Krebs solution. Segments of colon were opened along the mesenteric insertion and mucosal/submucosal layers were removed. Colonic samples were slitted along the longitudinal axis into strips of approximately 4 mm in width and 10 mm in length.

The preparations were set up in organ baths containing Krebs solution at 37° C., bubbled with 5% CO₂+95% O₂, and connected to isometric transducers (constant load=0.5 g). The mechanical activity was registered by BIOPAC MP150 (Biomedica Mangoni, Pisa, Italy). The Krebs solution was composed as follows: KCl 4.7 nM, NaCl 113 nM, KH₂PO₄ 1.2 nM, CaCl₂ 2.5 nM, MgSO₄ 1.2 nM, NaHCO₃ 25 nM, glucose 11.5 nM (pH 7.4±0.1). Each preparation was allowed to equilibrate for at least 30 minutes, with intervening washings at 10 minutes intervals. A pair of coaxial platinum electrodes was put at a distance of 10 mm from the longitudinal axis of each preparation to bring electrical stimulation by a BM-ST6 stimulator (Biomedica Mangoni, Pisa, Italy). At the end of equilibration period, each preparation was repeatedly challenged with electrical stimuli, and experiments started when reproducible responses were obtained (usually after two or three stimulations).

In the first set of experiments, the total electrical stimulation was recorded in colonic samples maintained in standard Krebs solution.

In the second and third set of experiments cholinergic contractions were recorded. Colonic samples were maintained in Krebs solution containing N-ω-nitro-L-arginine methylester (L-NAME, nitric oxide synthase inhibitor, 100 μM), N-acetyl-1-tryptophan 3,5-bis(trifluoromethyl) benzylester (L-732,138, neurokinin NK1 receptor antagonist, 10 μM), 5-fluoro-3-[2-[4-methoxy-4-[[(R)-phenylsulphinyl]methyl]-1-piperidinyl]ethyl]-1H-indole (GR159897, NK2 receptor antagonist, 1 μM), (R)-[[(2-phenyl-4-quinolinyl)carbonyl]amino]-methyl ester benzeneacetic acid (SB218795, NK3 receptor antagonist, 1 μM) and guanethidine (adrenergic blocker 10 μM) in order to assess the neurogenic contraction and in Krebs solution containing tetrodotoxin (TTX, 1 μM) and challenged with carbachol (CCh, 10 μM) to study the myogenic responses. The fourth and the fifth series of experiments were set up in order to assess the tachykinergic NK1 stimuli. The neurogenic NK1 contractions were recorded in colonic specimens maintained in Krebs solution containing L-NAME (100 μM), guanethidine (10 μM), GR159897 (1 μM), SB218795 (1 μM) and atropine sulphate (muscarinic receptor antagonist, 1 μM) whereas the myogenic activity was detected maintaining the specimens in TTX-added Krebs solution and stimulating with exogenous substance P (SP, 1 μM).

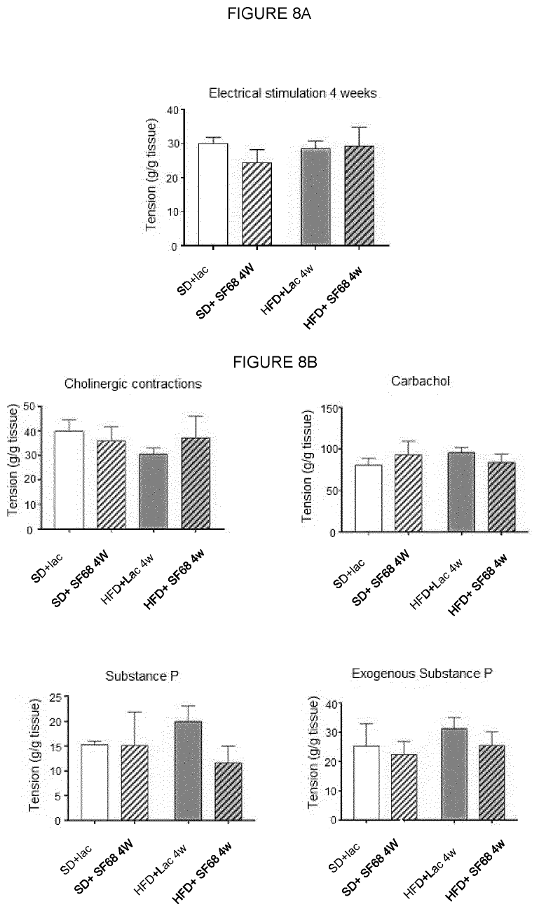

During the stabilization period of the colonic tissues maintained in standard Krebs solution, the preparations showed a rapid spontaneous motor activity which remained stable through the whole experiment and, in most cases, was minimal and did not interfere with the motor responses evoked by the electrical stimulus (ES). The electrically induced responses were characterized by phasic contractions followed, in some cases, by post-contractions of variable width. In colonic preparations from SD or HFD animals, maintained in standard Krebs solution, the application of electrical stimuli induced comparable contractile responses after 4 w of treatment with SD or HFD (FIG. 8A). In the analysis of the main ENS excitatory systems (acetylcholine and substance P), an evaluation of the endogenous and exogenous, cholinergic and tachykinergic contractile responses has been carried out and they do not result to be altered by HFD after 4 w or changed by SF68 at 4 w (FIG. 8B).

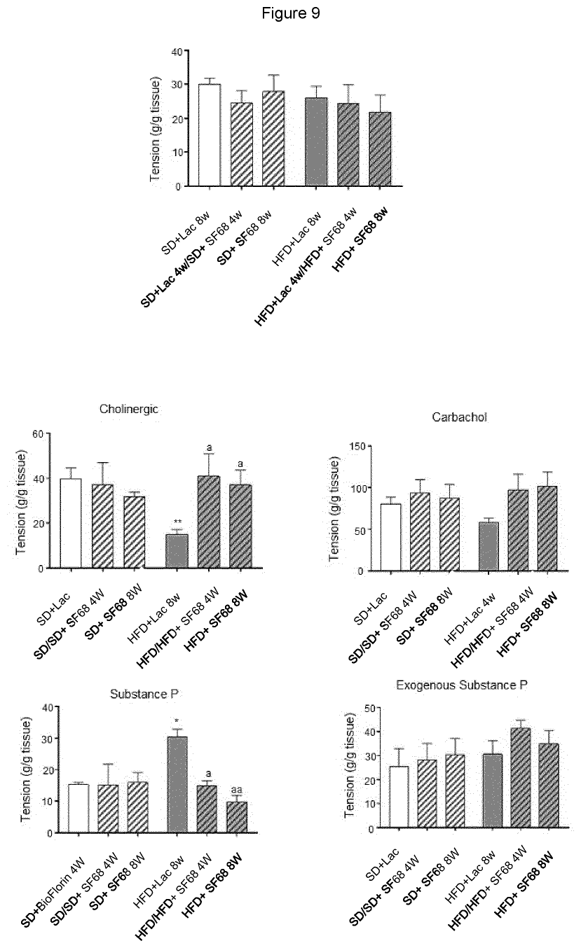

In colonic preparations from SD or HFD animals, maintained in standard Krebs solution, the application of electrical stimuli induced comparable contractile responses after 8 weeks of treatment with SD or HFD (FIG. 9). In the analysis of the main ENS excitatory systems (acetylcholine and substance P), we observed a decrease of cholinergic activity and an increase of tachykinergic activity in the animals fed with HFD after 8 weeks (FIG. 9). The treatment with SF68 for 8 weeks normalized the functional changes induced by the high-fat diet (FIG. 9).

The high-fat diet causes an alteration of the enteric nervous component. Obese people have “IBS-like” symptoms of constipation type, due to the alteration of peristalsis consisting in alternate inhibitory and stimulatory stimuli.

Contracting stimuli are acetylcholine, which lasts few milliseconds, and substance P (tachykinin), which has a longer action. Relaxing stimulus is nitrogen monoxide. Physiologically, the contraction is predominantly cholinergic while in inflammatory pathologies or HFD the contraction is predominantly tachykinergic.

HFD (and generally tissue inflammation) is associated with 1) decrease of cholinergic response and 2) increase of substance P, with the effect of prolonging the contraction of the longitudinal smooth muscles as compared to the digestive tract (constipation). SF68 is able to statistically significantly counteract both the increase of the tachykinergic component consequent to hypercaloric diet and the loss of cholinergic responses. It is worth noting that acetylcholine, in addition to be a neurotransmitter, carries out immunomodulator activity, decreasing the activities of immunoinflammatory cells.

The cholinergic contractions, which are the first to “ruin themselves” during inflammation, are stronger in the samples treated with SF68 after 8 weeks than in HFD control. Meanwhile substance P seems to decrease. These effects are canceled by the addition of tetrodotoxin, which blocks the neural component and therefore it does not seem that the effects of SF68 are at the muscle level but at systemic level affecting its neuroenteric components.

### Western Blot Analysis of Tight Junctions in Colonic Tissue

Colonic tissues were weighed and then homogenized in lysis buffer (50 mg in 400 μl), by using a Polytron homogenizer (QIAGEN, Milan, Italy). The homogenates were centrifuged at 12000 rpm for 15 min at 4° C. and the resultant supernatants were then separated from pellets and stored at −80° C. Bradford test was carried out to quantify the total proteins. Subsequently, the proteins were separated on a pre-formed 4%-20% polyacrylamide gel (Mini-PROTEAN TGX gel, Biorad) and transferred to PVDF membranes (Trans-Blot Turbo™ PVDF Transfer Packs, Biorad). The membranes were blocked with 3% BSA diluted in Tris-buffered saline (TBS, 20 mM Tris-HCl, pH 7.5, 150 nM NaCl) with 0.1% Tween 20. Primary antibodies against p-actin (ab8227, Abcam), occludin (ab167161, Abcam), zonulin-1 (ab96587, Abcam), claudin (ab15098, Abcam), toll-like receptor (TLR) 4 (ab22048, Abcam), TLR2 (ab213676, Abcam), NF-κB-p65 (sc-8008, Santa Cruz), MyD88 (sc-136970, Santa Cruz), sodium coupled monocarboxylate transporter 1 (SMCT1, BS-6106R, Bioss), monocarboxylate transporter 1 (MCT1, PA5-76687, Thermo Fisher), monocarboxylate transporter 4 (MCT4, PA5-106683, Thermo Fisher) were used. The secondary antibodies were purchased by Abcam (anti-mouse ab97040 and anti-rabbit ab6721). The protein bands were detected with ECL reagents (Clarity Western ECL Blotting Substrate, Biorad). The iBright analysis software was used to perform the densimetric analysis.

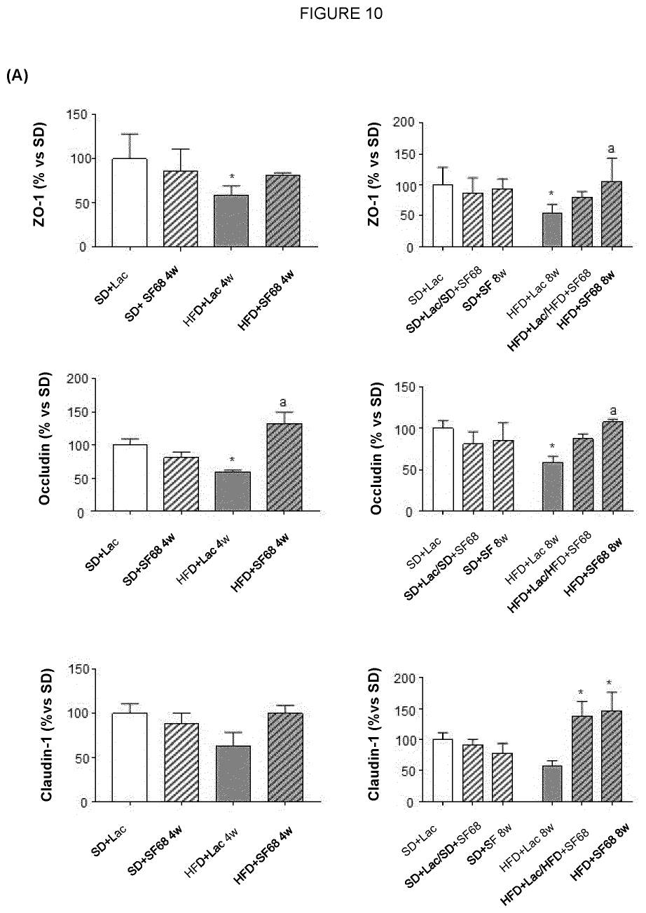

Feeding with HFD for 4 as well as for 8 weeks resulted in a significant decrease of the tight junction levels at the colonic tissue (FIGS. 10A, B and C). Supplementation with SF68 for 8 weeks resulted in a normalization of zonulin 1, occludin and claudin-1 (FIGS. 10A, B and C), while the treatment 4+4 w resulted in an improvement of the claudin-1 expression but not of zonulin 1 and occludin (FIG. 10C).

### Evaluation of Toll-Like Receptor Expression in Intestinal Tissue

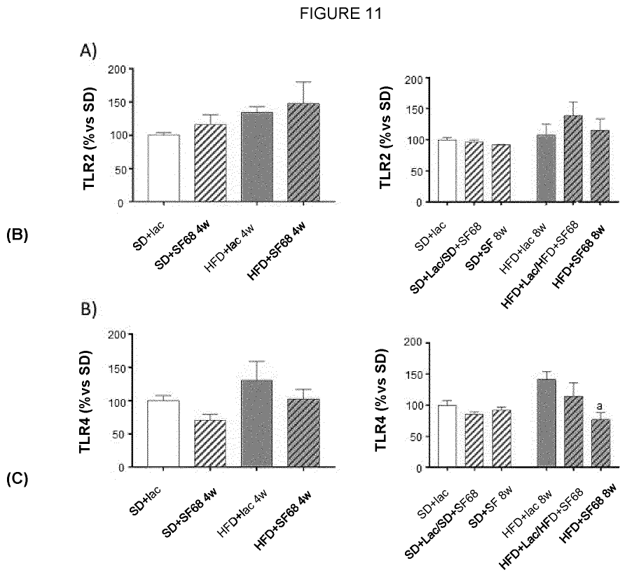

Animals fed with HFD both for 4 and 8 weeks did not show significant changes of the toll-like receptor colonic expression. The treatment with SF68 did not significantly change TLR2 levels (FIG. 11A). Supplementation for 8 weeks with the probiotic resulted in the significant decrease of TLR4 colonic levels compared to the group fed with HFD (FIG. 11B).

TLRs are a class of transmembrane receptor proteins mainly present on macrophages and dendritic cells playing a key role in natural immunity. TLRs, as a response to their link with ligands belonging to pathogens, activate the immune responses of the sentinel cells.

### Western Blot of the Molecular Signaling Paths Nuclear Factor kB (NF-kB) and Myeloid-Differentiation Primary Response-Gene 88 (MyD88)

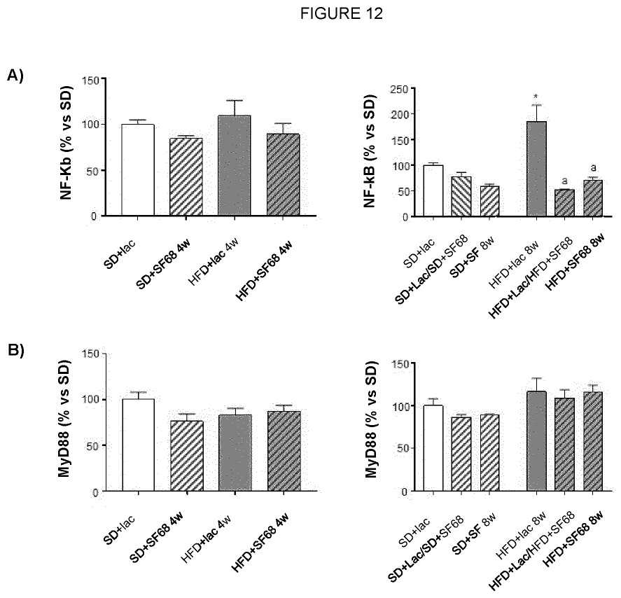

HFD administered for 8 weeks induced a significant increase of NF-kB levels in colonic tissues (FIG. 12A). The supplementation of the diet with SF68 resulted in the significant decrease of this parameter both in the 4+4 w and 8 w therapeutic scheme (FIG. 12A). Conversely, no significant changes among the different treatment groups were observed by analyzing MyD88 levels (FIG. 12B).

NF-kB plays a key role in the inflammatory response to infection and its increase has been associated to inflammation, cancer and autoimmune diseases.

### Immunohistochemistry and Immunofluorescence

Portions from formalin fixed full-thickness colonic samples were processed for immunoperoxidic staining, as described by Ippolito et al. [J. Cell. Mol. Med. (2015), doi:10.1111/jcmm.12428]. Sections were incubated with primary antibodies anti-claudin-1 and inflammatory infiltrate (Santa Cruz Biotech, California, USA) overnight at 4° C. and then exposed to suitable biotinylated immunoglobulins, complex of streptavidin labeled with peroxidase and 3,3′-diaminobenzidine (DakoCytomation, Glostrup, Denmark). Then, the immunostained sections were examined by a Leica DMRB microscope and representative photomicrographies were taken by a DFC480 digital camera (Leica Microsystems, Cambridge, UK) for quantitative evaluation.

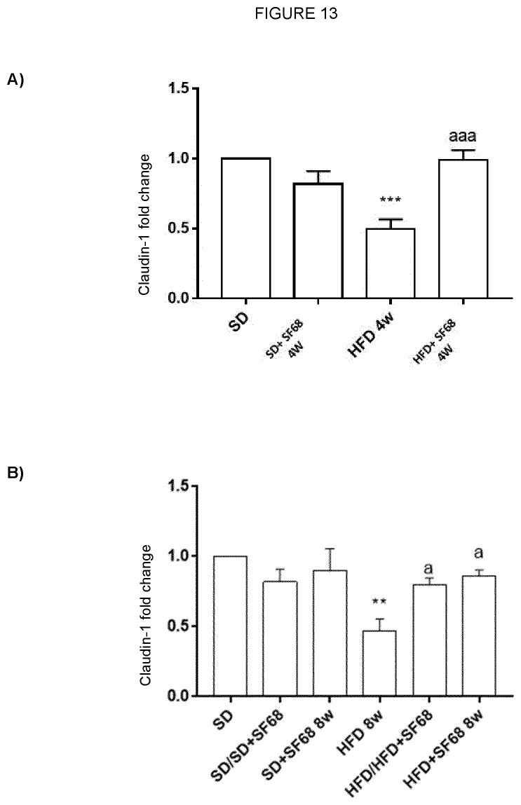

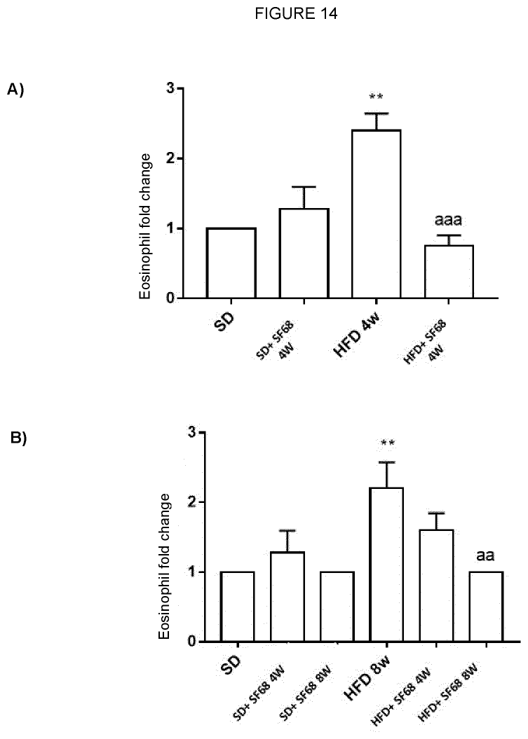

Feeding with HFD for 4 as well as for 8 weeks resulted in a significant decrease of claudin-1 levels (FIGS. 13A and B) and immunoinflammatory infiltrate (FIG. 14) at the colonic tissue. Supplementation with SF68 with the 4 w, 4+4 w and 8 w scheme resulted in a normalization of claudin-1 expression (FIGS. 13A and B) and a marked decrease of intestinal inflammation levels (FIG. 14).

### Evaluation of the Protein Expression of Butyrate Transporters

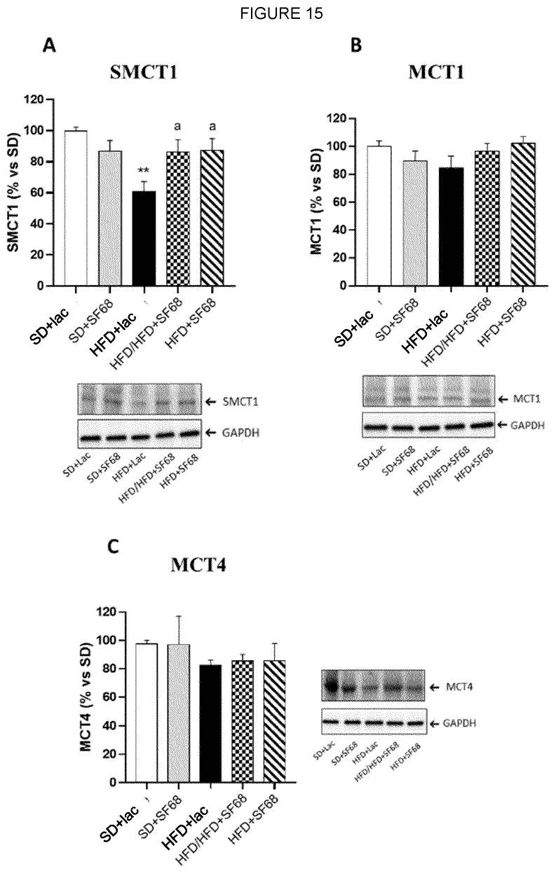

Mice fed with HFD for 8 weeks show a significant decrease in the apical transporter SMCT1, compared to SD control mice (FIG. 15A) while the levels of MCT1 (apical) and MCT4 (basolateral) transporters were not affected (FIGS. 15B and C). The treatment with SF68 for 4 and 8 weeks resulted in a normalization of the expression of the SMCT1 transporter in HFD mice (FIG. 15A), while no changes were observed on MCT1 or MCT4 protein expression (FIGS. 15B and C).

### Evaluation of the Citrate Synthase Activity on the Intestinal Mucosa

The frozen samples, resulting from the different mice groups, were homogenized on ice in a cold buffer (sucrose 250 mM, Tris 5 mM, EGTA 1 mM, Triton X-100 0.02%; pH 7.4) with GentleMACS dissociator (Miltenyi Biotec, Bologna, Italy). Homogenates were centrifuged at 12.000×g for 15 min at 4° C. (EuroClone, Speed Master 14 R centrifuge, Milan, Italy). The pellets were discarded and the supernatants used for protein quantification by Bradford assay and subsequently for the determination of the activity of citrate synthase enzyme.

The samples were diluted in Tris-buffer (100 mM; pH 8.2) containing 5,5′-dithiobis-2-nitrobenzoic acid (DTNB, 100 μM) and acetyl-coenzyme A (100 μM). The analysis was performed in 96 multi-well plates (1 μg of proteins per well) and the reaction started by the addition of a solution of oxaloacetic acid (500 μM). The reaction was followed spectrophotometrically at 37° C. every 30 seconds for 15 min at the wavelength of 412 nm (EnSpire, PerkinElmer, Waltham, MA, USA). Citrate synthase activity was determined by comparing the samples activity with a known concentration of the isolated enzyme (Sigma-Aldrich, St. Louis, MO, USA). Citrate synthase activity was expressed in mU/ml.

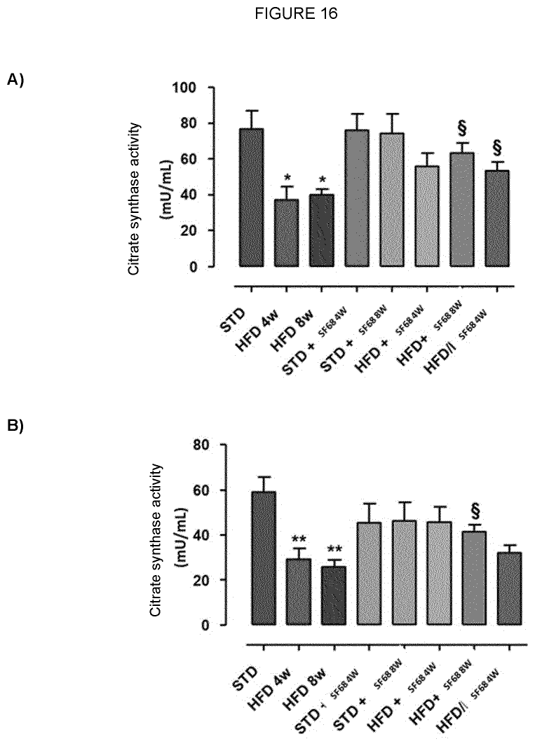

Mice fed with HFD for 4 or 8 weeks show a significant decrease in the citrate synthase activity at the intestinal mucosa and fatty tissue, compared to SD control mice (FIGS. 16A and B). The treatment with SF68 for 8 weeks resulted in a significant increase of the citrate synthase activity in the intestine and fatty tissue of HFD mice (FIGS. 16A and B).

### Evaluation of Plasmatic Homocysteine Levels

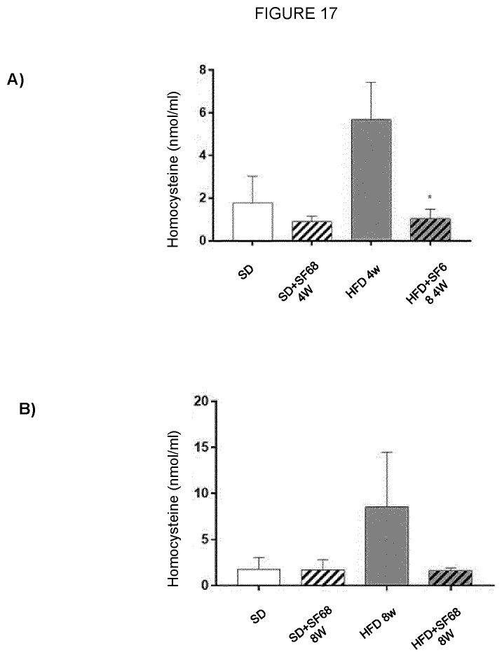

Animals fed with HFD showed an increase of plasmatic homocysteine levels (FIG. 17). The treatment with SF68 in mice under hypercaloric diet resulted in a significant decrease of the levels of this parameter in 4+4 w and 8 w scheme (FIG. 17).

Mice fed with HFD had an increase of plasmatic homocysteine levels, which may also occur in obese people under hypercaloric diet. The treatment with SF68 significantly reduced plasmatic homocysteine levels.

### Evaluation of TNF Levels

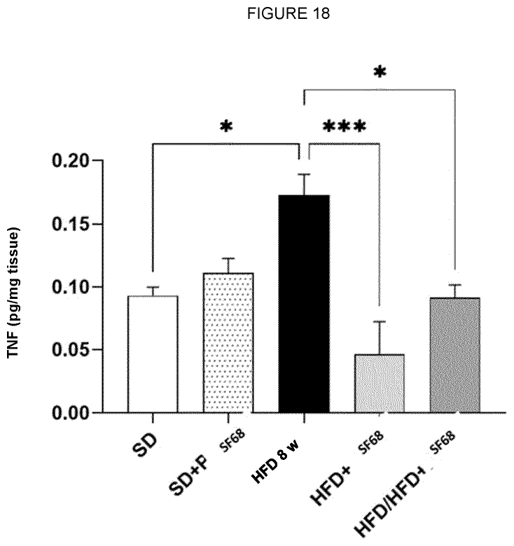

Animals fed with HFD showed an increase of TNF levels in brain tissues (FIG. 18).

The treatment with SF68 in mice under hypercaloric diet resulted in a significant decrease of the levels of this parameter in 4+4 w and 8 w scheme (FIG. 18).

### Statistical Analysis

The statistical analyses were carried out with GraphPad Prism 8 (GraphPad Software Inc, USA). Statistical significance was set at P value of 0.05.

For all the comparisons (SD vs HFD, SD vs SD*SF68, HFD vs HFD+SF68) the statistical significance was assessed by using t-Student test for uncoupled data.

1: A supplement based on Enterococcus faecium for use in the treatment of disorders characterized by alterations of the intestinal mucosa.

2: The supplement according to claim 1 wherein Enterococcus faecium is the strain with deposit number NCIMB 10415 which is the probiotic active ingredient called SF68©.

3: The supplement according to claim 1 wherein the disorder is inflammatory alterations of the intestinal mucosa.

4: The supplement according to claim 3 wherein the disorder is obesity.

5: The supplement according to claim 1, wherein Enterococcus faecium is used in combination with butyric acid or a salt thereof.

6: The supplement according to claim 5, wherein the butyric acid or salt thereof includes sodium butyrate or calcium butyrate.

7: A method of treatment of a disorder characterized by alterations of the intestinal mucosa, comprising the step of administering a supplement based on Enterococcus faecium.

8: The method according to claim 7, wherein the Enterococcus faecium is a strain with deposit number NCIMB 10415.

9: The method according to claim 7, wherein the disorder is inflammatory alterations of the intestinal mucosa.

10: The method according to claim 9, wherein the disorder is obesity.

11: The method according to claim 7, wherein the Enterococcus faecium is administered in combination with butyric acid or a salt thereof.

12: The method according to claim 11, wherein the butyric acid or salt thereof includes sodium butyrate or calcium butyrate.

13: The method according to claim 8, wherein the Enterococcus faecium is administered in combination with butyric acid or a salt thereof.

14: The method according to claim 13, wherein the butyric acid or salt thereof includes sodium butyrate or calcium butyrate.

15: The supplement according to claim 2, wherein Enterococcus faecium is used in combination with butyric acid or a salt thereof.

16: The supplement according to claim 15, wherein the butyric acid or salt thereof includes sodium butyrate or calcium butyrate.
